(19) United States
(12) Patent Application Publication
(10) Pub. No.: US 2008/0039921 A1
(43) Pub. Date: Feb. 14, 2008

(54) Balloon Catheter

(75) Inventors: Hans I. Wallsten, Denens (CH); Nora Brigitte Dognitz, Lausanne (CH); Jerome Duc, Corseaux (CH)

Correspondence Address: BUCHANAN, INGERSOLL & ROONEY PC, POST OFFICE BOX 1404, ALEXANDRIA, VA, 22313-1404, US

(73) Assignee: WALLSTEN MEDICAL S.A., Denens (CH)

(21) Appl. No.: 11/630,792

(22) PCT Filed: Jun. 9, 2005
(86) PCT No.: PCT/SE05/00869

§ 371 Date: Dec. 27, 2006

(30) Foreign Application Priority Data
Jun. 30, 2004 (SE) 0401708-3

(51) Int. Cl. A61F 2/84; A61F 2/04; A61F 7/12

(52) U.S. Cl. 623/1.11; 604/113; 623/23.66

(57) ABSTRACT

A balloon catheter is provided for the implantation of a stent in a mammalian duct or cavity, comprising an elongate distal section and an expandable first balloon accommodating the section, further comprising means for the supply of a pressure medium for the expansion of the balloon, and means for heating the pressure medium, the catheter being provided with an elongate stent mounted onto the balloon. The catheter contains second means for establishing outwardly directed expansion of the stent at a sight or location selected from the two ends of the stent so that the stent will remain in position as implanted after removal of the catheter from the duct or cavity; and a method for the implantation of a stent in a human prostatic urethra.

### FIELD OF THE INVENTION

This invention relates to temporary stents and particularly to the treatment of benign prostatic hyperplasia.

### BACKGROUND OF THE INVENTION

Almost all available stents are so called permanent stents e.g. they are designed to be inserted in occluded vessels and organs to permanently support the vessel and keep it open.

The majority of the permanent stents are expandable to facilitate insertion through a small access and the ratio of the non-expanded diameter to the expanded diameter which is determined by the size of the vessel is often small such as 1:3 and less. In general such stents have the openings in the walls to enhance overgrowth and ingrowth of tissue therefore the removal of expanded stents designed for permanent implantation is difficult and risky.

The golden standard for the treatment of benign prostate hyperplasia, BPH is transurethral resection TURP, which is surgery and means several days of hospitalisation and often results in severe side effects. It also means carrying an indwelling catheter during weeks and sometimes more.

Thermotherapy, such as heat treatment of the prostate with microwaves, interstitial radiofrequency, hot balloons are interesting alternatives to TURP because it is less invasive and have less side effects. It is accomplished by heating the prostate to temperatures above 50-60°, which leads to a decrease in the swelling of the prostate, as the prostatic gland.

A great problem connected to different kind of thermotherapy is however that the prostatic gland temporary swells in response to the burn so that at higher temperatures and more efficient treatment there is a great risk for acute blockage of the urethra.

During the healing process of the prostate and the urethra the damaged tissue will be reabsorbed and partly slough off. The healing takes several weeks and it is therefore necessary to catheterise the patient during the healing, which is a great drawback to this kind of therapy. A long catheterisation time means not only a great discomfort for the patient but also risk for infections. Thermotherapy will therefore hardly replace TURP unless the catherisation can be reduced to a few days or totally replaced.

It is therefore a need for a temporary stent to keep the urethra open during the healing. Such a stent must have an appropriate length and be precisely placed between the bladder neck and the outer sphincter to eliminate the risk for blockage by the swelling prostate on unsupported parts of the urethra or incontinence if a part of the stent protrudes into the sphincter. Such a temporary stent must also be easy to take out after the healing or in case of dislocation.

A commonly used material for stents is a so called memory or recovery metal such as Nitinol®, which is an alloy based on nickel and titanium. Such alloys undergo a transition between a strong austenitic state and a soft martensitic state at certain temperatures. They have been subject to a great deal of interest due to the extraordinary “memory” they possess.

An expanded memorised shape can be set into such recovery metals by heating them while they are constrained in the desired expanded configuration. The forming temperature for setting the initial shape is typically around 500° C. After cooling the alloy to its martensite state it can be mechanically deformed to a second, smaller configuration which is suitable for introduction in the organ to be stented. After placement is the alloy heated to its transition temperature, and expands to its austenite, strong state and recover its initial memorised configuration.

For replacement of indwelling catheters in urology a stainless steal coil was introduced by Fabian 1980 followed by several similar stents for temperature use such as Prostakath™. A drawback with this type of non-expandable stents is that they often migrate.

Memokath®, Engineers & Doctors, Denmark was developed to overcome the shortcomings of Prostakath™. It is a cylindrical coil stent made up of a single wire of a Nitinol® alloy. The material has a transition temperature of about 45° C. and becomes soft at 10° C. Because of the memory capability the stent is given a primary shape where one or more segments has a diameter, which is considerably greater than the rest of the stent and a secondary shape where the segment with the greater diameter has been reduced to the same smaller diameter as the rest of the stent, WO 93/13824 and “From Prostakath® to Memokath®”, Nordling et al. in “Stenting the Urinary System”, pp. 285-290, Oxford Isis Medical Medica Ltd, 1998.

In the commercial configuration has the Memokath® stent in its non-expanded secondary shape an inner diameter of 6.7 mm. In the primary shape is only the proximal part adjacent the sphincter expanded to a bell shape with tightly coiled wires. The expansion is activated by the injection of water at 50 to 60° C., expanding the proximal part to a diameter of about 13 mm locking the stent in the urethra. The expansion takes place by the unwinding of the outer coil and then coil by coil successively until the expansion is accomplished. The injection of a hot liquid into the urethra can, however, cause pain and can even damage certain parts of the urethra, such as the sphincter. If lower temperatures are used, there is on the other hand a risk that the stent does not develop fully.

The stent can be taken out by injection of cold ice water so the material reverts to its pliable, soft martensite state allowing deformation so the stent spiral will be partly straightened when it is pulled out using the grasping forceps of a cystoscope.

It has also been proposed the use of biodegradable polymers for temporary stents. Such a stent should be strong enough to support the wall during several weeks and then degrade.

Suitable material for such stents are for example polymers of poly-k-hydroxiacids such as polyglycolic acid (PGA) or polylactic acid (PLLA) used since many years in bone surgery. These materials are thermoplastic and can be thermoformed or drawn in the form of strips or wires.

Stents made of such materials have been suggested as replacement for permanent, metallic stents for stenting of coronary arteries to prevent restenosis after coronary intervention, which commonly occurs 3 to 6 months afterwards. So for example is the Igaki-Tamai stent made of PLLA in a zigzag helical coil pattern. The stent is expanded by inflation of a standard angioplasty balloon catheter with a heated liquid (Tamai et al: “Initial and 6 months results of Biodegradable Poly-1-Lactid acid coronary stents in human). It is important that such an expansion is almost immediate and complete along the entire length of the stent to get fixation of the stent and to prevent occlusions in the coronay vessels and minimise vessel injury by heat. This is possible because the expansion occurs by straightening of the zigzag formed helical design.

It has also been proposed to use biodegradable polymers in the form of a spiral as a temporary stent for the treatment of BPH. An example is the Spiroflo® stent (Mentor, USA) which is made of a copolymer of PGA and PLLA (PLGA). It would be that there is no need for a second intervention to take out the stent as for the metallic stents. This spiral has an initial outer diameter of about 8 mm and can be inserted with the help of a cystoscope. Although, such stents can self-extend somewhat after insertion due to influence of the body temperature, the expansion is slow and takes several days. The expansion is also too small to allow a good fixation against the urethra. For the use after treatment of BPH with pressure and heat it would be desirable with high radial expansion rates of 2 to 3 times of the stent to get a good fixation.

In pending application PCT/EP01/05544 there is disclosed the use of a balloon catheter for the treatment of BPH by heat and pressure and a subsequent implantation of stent of a degradable material. The disclosure if this pending application is incorporated herein by reference.

According to one embodiment in said PCT/EP application the stent is constituted by a spiral produced from a straight filament and wound to a spiral with a large diameter and then wound in cold condition to a spiral of a small diameter for example 5 mm. Such a stent mounted on a balloon catheter expands by itself by heating to a diameter depending of the temperature, for example to 12 mm at a temperature of 50° C. (p. 20, col. 12-29).

Further trials have however shown that there are several drawbacks related to this embodiment. The expansion of the stent starts from the two end coils of the stent, which rotate in opposite direction and continue coil by coil. The expansion is counteracted by the friction between the balloon and the coils and the friction increases with increased number of coils. The expansion is therefore slow and does not allow the expansion of longer stents or stents with large expansion rates. Yet another drawback is that if a large expansion is desired the stent will shorten considerably.

### OBJECTS AND SUMMARY OF THE INVENTION

The main object of the present invention is to provide a balloon catheter for the implantation of a stent in a mammalian duct of cavity. The term “mammalian” is intended to cover implantation on humans.

Another object of the invention is to provide a balloon catheter capable of implantation of a stent in a mammalian duct or cavity so that the stent will reach a correct position and remain in this position as implanted after removal of the catheter per se.

Yet another object of the invention is to provide a method for safe implantation of a stent in a human prostatic urethra using a balloon catheter according to the invention.

A further object of the invention is to provide a balloon catheter for the implantation of a stent and a method associated therewith, said catheter being provided with a positioning balloon attached at the front end of the catheter for correct positioning of the catheter before the implantation of the stent.

Another object of the invention is to provide a balloon catheter for the implantation of a stent, wherein outwardly directed pressure is combined with heating for the fixation by local expansion of the stent.

A further object of the invention is to provide a balloon catheter for the implantation of a stent and a method associated therewith, wherein a heated fluid is introduced and circulated under pressure through the balloon for the expansion and fixation of the stent, thereby avoiding uncontrolled spillage of the hot fluid in the mammalian duct.

Still another object of the invention is to provide a balloon catheter capable of implantation of a stent having inherent memory properties.

A further object of the invention is to provide a tubular spiral stent with means for easy removal of the stent when implanted in a mammalian duct or cavity.

Accordingly, a first aspect of the invention is the provision of a balloon catheter for the implantation of a stent in a mammalian duct or cavity, said catheter comprising an elongated distal section and an expandable first balloon accommodating said section, further comprising means for the supply of a pressure medium for the expansion of said balloon, and means for heating said pressure medium, the catheter being provided with an elongated stent mounted onto said balloon. The catheter further comprises second means for establishing outwardly directed local expansion of the stent at a site or location selected from the two ends of the stent, so that the stent will remain in position as implanted after removal of the catheter from the implantation site.

It is preferred that said second means is constituted by said stent being shorter than the expandable part of said balloon so as to provide for local expansion of said stent at least at one end thereof.

A second aspect of the invention resides in a balloon catheter wherein a sleeve is placed between the balloon and the stent.

A third aspect of the invention resides in a balloon catheter, wherein said sleeve and said stent form an assembly.

A fourth aspect of the invention resides in the use of a stent having memory properties for assistance of its local expansion.

A fifth aspect of the invention resides in a method for the implantation of a stent in a human prostatic urethra using a balloon catheter having a balloon for positioning in the urethra, comprising the steps:

a) applying a stent of selected length capable of local radial expansion in a predetermined axial position onto said balloon;

b) inserting the catheter thus prepared into said urethra for positioning the stent therein;

c) distending the balloon by introducing a heated pressurized fluid therein to expand the stent at one or both ends thereof so as to keep the stent in position in said urethra;

d) removing fluid from the balloon and withdrawing the catheter from the urethra leaving the stent in position as implanted.

A sixth aspect of the invention resides in a method combining treatment of a prostate by heat and subsequent implantation of a stent, said method using a balloon catheter having a treatment balloon matching the relevant treatment zone and also having a distal positioning balloon to be placed in the urinary bladder, comprising the followings steps:

1) measuring the relevant treatment length of said urethra;

2) introducing the catheter into said urethra;

3) expanding said positioning balloon and retracting the catheter to engagement of said balloon against the bladder wall to obtain correct position of the treatment balloon;

4) distending the treatment balloon by introducing a heated pressurized fluid therein to provide dilation and heat treatment of the prostate urethra;

5) deflating both balloons and retracting the catheter from the urethra;

6) while using the same catheter, applying a stent of a selected length capable of local radial expansion in a predetermined axial position onto said treatment balloon;

7) inserting the catheter thus prepared into said urethra;

8) expanding said positioning balloon and retracting the catheter to engagement of said balloon against the bladder neck to obtain correct position of the treatment balloon;

9) distending the treatment balloon by introducing a heated pressurized fluid therein to expand the stent at a site or location selected from the two ends of the stent so as to keep the stent in position in said urethra; and

10) removing fluid from the balloons and withdrawing the catheter from the urethra leaving the stent in position as implanted.

Further features and details of the balloon catheter according to the present invention are clear from the dependent further claims as appended hereto.

In this disclosure the expressions “distal” and “proximal” are used with the meaning “front” and “rear”, respectively, i.e. related to the operator of the catheter. Furthermore, by the expression “at a site or location selected from the two ends of the stent” is meant one or both ends of the stent.

### DETAILED DESCRIPTION OF THE INVENTION

The invention will in the following be further described by exemplifying embodiments which, however, must not be construed to restrict the scope of protection except as defined in the appended claims. These embodiments are described with reference to the appended drawings, wherein:

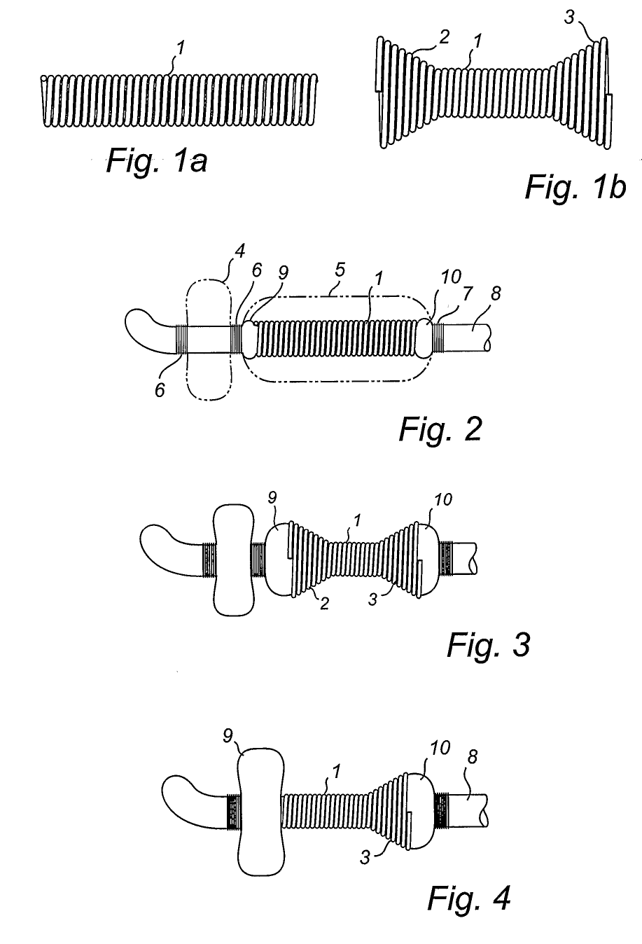

FIGS. 1a, 1b show two different configurations of a stent operating in accordance with the present invention;

FIG. 2 shows more in detail the front part of a balloon catheter provided with a positioning balloon and a stent;

FIG. 3 shows a detail of the front part shown in FIG. 2 with a stent in an expanded configuration at both ends;

FIG. 4 shows an alternative front part arrangement with only proximal expansion of the stent;

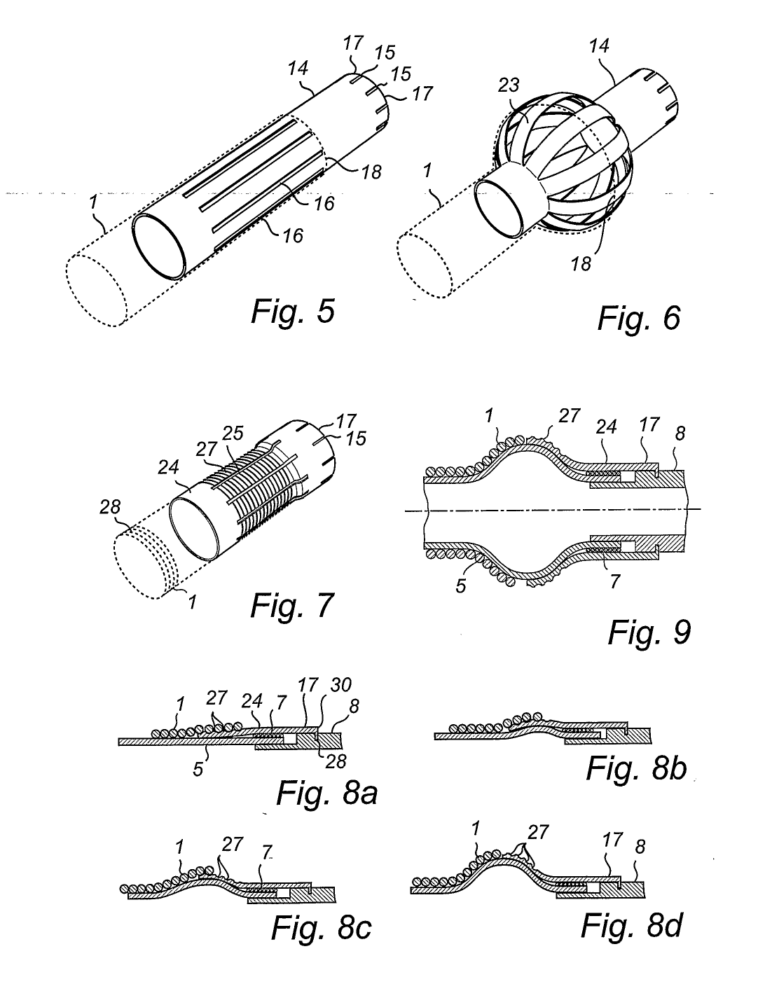

FIG. 5 shows another embodiment of the catheter of the invention in form of a sleeve;

FIG. 6 shows the embodiment of FIG. 5 in an expanded configuration;

FIG. 7 shows yet another embodiment of the catheter of the invention;

FIGS. 8a-8d show more in detail the progression of expansion of one end of a stent;

FIG. 9 shows more in detail the expanded configuration of the embodiment of FIGS. 7 and 8;

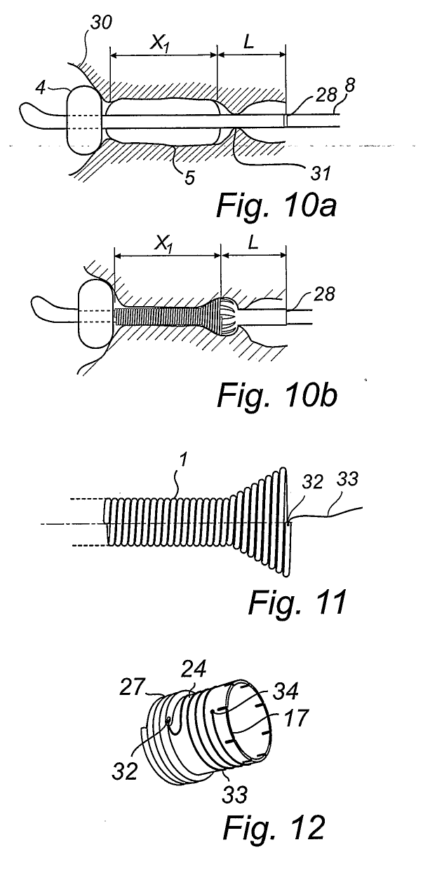

FIGS. 10a and 10b illustrate diagrammatically two subsequent steps of treatment of a prostate by heat and subsequent implantation of a stent;

FIG. 11 shows an implanted stent provided with means for simple withdrawal of the stent after implantation thereof; and

FIG. 12 shows a sleeve carrying the distal part of the stent and a thread wound onto the sleeve intended for removal of the stent upon implantation thereof.

FIGS. 1-2 show one preferred embodiment of the invention suitable as a stent for a treatment of BPH.

FIG. 1 shows a spiralshaped stent 1 in two configurations; FIG. 1a before implantation and FIG. 1b after implantation and expansion of both ends 2,3 by heat and pressure according to the invention.

The material can be a recovery memory metal, such as Nitinol®. In this case the wire is first wound around a tool with the same configuration as is shown in FIG. 1b. The package is then heated to the forming temperature as earlier described. After cooling to the martensite state the softened material is wound around a shaft to the configuration as shown in FIG. 1a.

The material can also be a thermoplastic material in form of a non-degradable material or a biodegradable polymer, such as the copolymer of PGA and PLLA mentioned above. In the case of a polymer a memory effect can be used in the following manner.

The wire is first wound around a tool with the same configuration as is shown in FIG. 1b. The package is then heated to a forming temperature for setting the initial shape which is below the melting temperature of the material. After cooling the stent has taken the shape as shown in FIG. 1b and has memorised same. The stent is then softened at a temperature just above the glass transition temperature and wound around a shaft of the same configuration as shown in FIG. 1a and then cooled. Examples of forming temperatures for the initial shapes are about 70-120° C. and softening temperatures are about 45-60° C.

The outer diameter of stent 1 according to FIG. 1a should not exceed about 7 to 8 mm to facilitate the implantation through the penile urethra. The inner diameter shall not be too small to prevent obstructions in form of blood clots or sloughing tissue. If possible the inner diameter should not be below 5 mm. Important therefore is the thickness of the selected filament or wire which could be round or have a flat configuration.

The spiralshaped stent has many advantages. It is easy to manufacture and allows easy removal. FIG. 1b depicts the stent 1 with the two ends 2 and 3 expanded. The expansion according to the invention is fast and almost immediate which will be explained later and will have the purpose to give a firm fixation of the stent in the tissue immediately after the positioning thereof.

Trials have shown that the enlarged diameter of the ends after thermotherapy should be in the range of about 11-13 mm which means a doubling compared to the diameter of the non-expanded stent body.

For the insertion and expansion of the stent, according to FIG. 1a, in this embodiment a device in form of a balloon catheter can be used as described in pending application PCT/EP01/05544 and particularly shown in FIG. 3 and FIG. 8. However other types of insertion/expansion devices can be used as explained later.

FIG. 2 shows such a balloon catheter having an inflated distal balloon 4 for positioning of the catheter during the thermal treatment of the prostate and an inflated treatment balloon 5. For fixation of balloons 4 and 5 sutures 6 and 7, respectively, are used. FIG. 2 also shows a catheter shaft 8. As described in application PCT/EP01/05544 the treatment balloon is first adjusted to the desired treatment length. After introduction of the catheter the position of the treatment balloon is controlled by using the positioning balloon 4, which during the treatment is maintained in position by retracting the catheter balloon 4 against the bladder neck by proximal pulling of the catheter.

For the insertion and fixation of a stent according to the invention a spiralshaped stent is selected which has a length corresponding to the active length of the inflated treatment balloon 5. The length could be somewhat shorter than the distance between the sutures 6 and 7. The stent 1 is positioned symmetrically between the two sutures 6 and 7. To firmly hold the stent 1 in place the treatment balloon 4 is partially inflated with liquid. If a shorter stent is used the two ends 2 and 3 will form two small bulges 9 and 10, respectively. The device can now be introduced into the urethra and positioned with the help of the inflated positioning balloon as described in the above-mentioned pending application.

The liquid in the system is then fully pressurised and the circulation and heating started. As there is a small quantity of liquid in the system the heating-up time is short and could be less than one minute. When the temperature of the stent is somewhat higher than the transition temperature in the case of using a Nitinol® material or higher than the softening point or glass transition point in the case of using a polymeric material the expansion starts by rotation of the outer turns of the stent ends 2 and 3 and at the same time the diameter of these turns will increase. Examples of preferable temperatures are about 65 to 75° C. for a Nitinol® stent with a transition at about 45° C. The use of a closed system with the hot liquid for the expansion of the stent has also the advantage that there is no risk for damages caused by the uncontrolled spilling of the liquid in the urethra.

Due to pressure the hot balloon will also expand correspondingly and keep the rotating turns hot by heat conduction. The expansion will continue until the stent ends 2 and 3 have expanded to the configuration as shown in FIG. 3.

It is an advantage if the two expanded ends 2 and 3 are equal in size and shape. Trials with the use of different shape memory materials, such as Nitinol® wires or polymers in the form of non-degradable materials have shown that at expansion by heat as described above the two ends expand equally.

According to a preferred embodiment of the invention only one end of the stent is expanded. This can be achieved if the stent is positioned on the device as depicted in FIG. 4 with its proximal end pushing against the positioning balloon 9. At inflation and heating the proximal end of the stent 1 will expand as the bulge 10 is developed. The stent 1 will be forced forward because of the pressure of the bulge 10 and the positioning balloon 9 will serve as a stopper.

The embodiments with a single expanded end has several advantages. One is that less shortening of the stent will occur. Another advantage is that only the proximal end 3 is memorised to expand. Consequently such a spiral stent can be cut to the desired length by cutting the distal end of the stent. Therefore a relatively long stent could be used and adapted to several stent lengths by cutting. The embodiments described above are particularly suitable for reinforced balloon catheters as disclosed in pending application PCT/EP01/05544 because there is no risk for bursts of the bulge because of the reinforcement.

FIG. 5 shows another preferred embodiment of the invention according to which there is no need for reinforced balloons. A sleeve 14 of a thin material with low elasticity and low surface friction, such as Teflon®, is shown. The sleeve 14 is to be positioned over the deflated balloon of a balloon catheter with pressure and heating means. In the proximal end of sleeve 14 there are several slits 15 forming flexible flaps 17 which could be arranged for fixation of the sleeve to the catheter shaft in a way which will be explained in another preferred embodiment. A number of other slits 16 are also arranged around the sleeve 14.

For the mounting of a stent 1 shown with dotted lines sleeve 14 is first positioned over the deflated balloon and fixed to the catheter shaft 8 with the flexible flaps 17. The stent 1 is then positioned over the sleeve 14 with the proximal end 18 juxtaposed to the proximal end of the slits 16.

FIG. 6 shows how the central part of the sleeve 14 expands when the balloon is expanded and heated by a pressurised medium, e.g. a liquid. The strips 23 formed between the slits 16 expand to a bulge exercising a pressure and transfer heat for simultaneous expansion of the proximal stent end in a similar way as shown in FIG. 4. As almost all expandable balloons are made of highly elastic materials, such as latex and silicone, their surfaces have a high friction. The use of an intermediate sleeve of a low friction material, such as Teflon®, will considerably facilitate the expansion of the stent. The balloon can also be made of material without reinforcement as it is totally enclosed by the stent body and the sleeve 14. The elastic balloon will by its expansion prevent the stent from movement in an axial direction. The sleeve 14 can also be cut in suitable lengths to fit the stent.

FIG. 7 shows another preferred embodiment, which is a modification of the device shown in FIGS. 5 and 6. A sleeve 24 of a thin material with low elasticity, low friction and good thermal conductivity is shown. The proximal end of the sleeve 24 has a number of slits 15 and corresponding flexible flaps 17, as shown in FIGS. 5 and 6. In the sleeve 24 a number of slits 25 are arranged. A threading 27 is arranged on the surface of the sleeve 24. The threading 27 corresponds to the winding 28 of a spiral stent 1 so that the stent end can be attached to the sleeve by screwing. In such a way the stent 1 and the sleeve 29 form one assembly, which can be positioned over the deflated balloon and attached to the balloon catheter as earlier shown in connection with the embodiment according to FIGS. 5 and 6.

A detail of the arrangement is shown in FIGS. 8a-d, which shows step by step the expansion of the end of the stent and the sleeve 24 at expansion of the heated balloon 5.

In FIG. 8a there is shown a part of the treatment balloon 5, the sutures 7 for fixation of the balloon to the catheter shaft 8, and part of the stent 1. Further a part of the sleeve 24 is shown with the end of the stent screwed onto the threading 27. For fixation of the assembly to the catheter 8 a recess 28 is arranged in the catheter shaft. The ends 30 of the elastic flaps 17 are bent inwards, so as to be received in the recess 28 when the unit stent sleeve is positioned over the balloon catheter for fixation of the assembly to the catheter shaft.

FIGS. 8b, 8c and 8d show in sequences the simultaneous expansion at the end of the stent and the balloon 5. In FIG. 8b there is shown how the expansion starts at the last windings of the end of the stent which unwind guided by the threadings 27. FIG. 8c shows the end of the stent partly expanded, and FIG. 8d shows the fully expanded end of the stent, which is released from its fixation to the sleeve 24 and consequently also the catheter.

FIG. 9 shows a part of the assembly corresponding to the stent/sleeve detail shown in FIG. 8d after the expansion of the stent.

This embodiment has many advantages. One is that the stent can be delivered from the factory mounted on the sleeve forming one single unit.

FIGS. 10a and 10b illustrate diagrammatically the two subsequent steps of treatment of a prostate by heat (FIG. 10a) and the following positioning and implantation of a stent according to the invention (FIG. 10b). FIG. 10a shows a treatment catheter as for example disclosed in PCT/EP01/05544 but the catheter can be any other suitable catheter known in the art. FIGS. 10a and 10b show details of a catheter having a shaft 8, a positioning balloon 4, a bladder neck 30, and expanded treatment balloon 5, the internal sphincter 31 and recess 28 in the catheter shaft 8.

As described above the physician first selects a balloon of appropriate length for the heat treatment, the length corresponding to that of the treatment zone depicted X₁. This length can for example correspond to the distance from the bladder neck to the veru montanum. The distance between the proximal end of treatment zone and the recess 28 on shaft 8 is depicted L in FIG. 10. After concluded treatment the catheter is taken out after deflation of balloons 4,5, and the physician has simply to select an assembly of a sleeve and a stent of desired length, the latter preferably of an expanded length corresponding to the length X₁ of the treatment zone. Since in accordance with the invention stent assemblies are provided with different stent lengths but with the same length L the stent will be implanted in a correct position in the treatment zone (FIG. 10b).

As an alternative for the implantation of a stent after a heat treatment of any kind one can provide a simple balloon catheter with a stent sleeve assembly, wherein a fluid, such as water, is externally heated. The heated fluid is introduced, such as by a syringe, into a central catheter tube causing expansion of the balloon and the stent, such as by a restricted outlet. During the expansion of the stent by the hot fluid it will leave the catheter via said outlet. This procedure will take only about 30 seconds as the fluid is preheated. By using a closed system of hot liquid expanding a balloon there is no risk for spilling liquid causing damages.

FIG. 11 shows an embodiment according to the invention for facilitating the removal of a stent after implantation. A flexible thread 33 is attached to the proximal end of the stent 1, such attachment being made for example through a hole in the stent end 32. When the stent is in position as implanted the thread 33 is passing through the closed sphincter and floats freely in the urethra. When the stent is to be removed the thread can be easily grasped by an endoscope. After injection of cold water the stent will soften when reaching the martensite state and the stent can be removed by first pulling the thread into the channel of the endoscope and then retracting the endoscope together with the attached stent, thereby eliminating the risk for scratching the inside of the urethra.

As described above the stent end rotates at extension thereof (FIGS. 8 and 9) and a thread attached according to FIG. 11 can therefore prevent the unwinding of the stent. The arrangement according to FIG. 12 can solve this problem.

FIG. 12 shows the proximal part of an assembly including a sleeve 24 and the proximal part of a stent 27 with the stent end 32 and the attached thread 33. As can be seen from the figure the stent is tightly wound in one direction, whereas the thread is wound onto the sleeve 24 in the opposite direction. In such a way stent 27 and thread 33 will unwind simultaneously during expansion. The opposite end 34 of the thread 33 is detachably fixed to the sleeve 24 by suitable means, such as a glue. When the stent has expanded and the thread is unwound the catheter with sleeve 24 can therefore be removed and the stent left with the thread freely floating downstreams in the urethra. As a preferred feature the distal part 24 of the sleeve can be provided with threads or grooves to accommodate the thread 33 in the grooves.

It is to be noted that many variations of the invention as described are conceivable and within the skill of the artisan, and the invention is to be limited solely by the scope of the appendid claims. Thus, for example, different positioning means can be used, for the correct placement of catheter and stent. Instead of the use of a positioning balloon other means known in the art can be used, such as ultrasound.

1. Balloon catheter for the implantation of a stent in a mammalian duct or cavity, comprising an elongate distal section and an expandable first balloon accommodating said section, further comprising means for the supply of a pressure medium for the expansion of said balloon, and means for heating said pressure medium, the catheter being provided with an elongate stent mounted onto said balloon wherein second means for establishing outwardly directed local expansion of the stent at a site or location selected from the two ends of the stent so that the stent will remain in position as implanted after removal of the catheter from said duct or cavity.

2. Balloon catheter according to claim 1, wherein said second means is constituted by said stent being shorter than the expandible part of said balloon so as to provide for local expansion of said stent at least at one end thereof.

3. Balloon catheter according to claim 1, wherein said second means is constituted by a sleeve of a material of restricted elasticity and a low friction placed between the balloon and the stent, said sleeve being provided with slits or openings providing for local expansion sites for the stent.

4. Balloon catheter according to claim 1, wherein said second means is constituted by a preshaped balloon having at least one local site establishing local expansion of the stent when expanded.

5. Balloon catheter according to claim 1, wherein said stent is in the form of a spiral, a tube having slits or openings therein, or a braid.

6. Balloon catheter according to claim 1, wherein the stent is of a material having memory properties.

7. Balloon catheter according to claim 6, wherein said material is constituted by a recovery metal.

8. Balloon catheter according to claim 6, wherein said material has visco-elastic memory properties.

9. Balloon catheter according to claim 8, wherein said material is biodegradable.

10. Balloon catheter according to claim 8, wherein said material is selected from PGA, PLLA, PLA, and PLGA.

11. Balloon catheter according to claim 6, wherein for assistance of said local expansion the memory properties of said material are utilized at corresponding local sites.

12. Balloon catheter according to any preceding claim claim 1, for use in the prostatic urethra, further comprising a positioning balloon placed distally of said first balloon and positioned inside the urinary bladder, said positioning balloon being expandable by introduction therein of said pressure medium or a separate pressure medium.

13. Balloon catheter according to claim 1, wherein said first balloon is made of an elastic material.

14. Balloon catheter according to claim 3, wherein said stent is of spiral configuration and is threaded onto the distal end of said sleeve.

15. Balloon catheter according to claim 14, wherein the proximal end of said sleeve is provided with attachment means anchoring in a fixed axial position the sleeve to the distal section of the catheter.

16. Balloon catheter according to claim 3, wherein said sleeve together with a stent of appropriate length form an assembly before being mounted onto the catheter in preparation for implantation.

17. Balloon catheter according to claim 16, wherein said assembly is provided with a thread wound around said sleeve in opposite direction to the stent spiral, one end of said thread being attached to the distal end of the stent, and the other end of said thread being detachedly attached to the distal part of said sleeve.

18. A method for the implantation of a stent in a human prostatic urethra using a balloon catheter having a balloon for positioning in the urethra, comprising the steps:
a) applying a stent of selected length capable of local radial expansion in a predetermined axial position onto said balloon;
b) inserting the catheter thus prepared into said urethra for positioning the stent therein;
c) distending the balloon by introducing a heated pressurized fluid therein to expand the stent locally at one or both ends thereof so as to keep the stent in position in said urethra;
d) removing fluid from the balloon and withdrawing the catheter from the urethra leaving the stent in position as implanted.

19. A method for the treatment of and the implantation of a stent in a human prostatic urethra using a balloon catheter having a treatment balloon matching a treatment length to be measured and also having a distal positioning balloon to be placed in the urinary bladder, said method comprising the steps:
1) measuring the relevant treatment length of said urethra;
2) introducing the catheter into said urethra;
3) expanding said positioning balloon and retracting the catheter to engagement of said balloon against the bladder neck to obtain correct position of the treatment balloon;
4) distending the treatment balloon by introducing a heated pressurized fluid therein to provide dilation and heat treatment of the prostatic urethra;
5) deflating both balloons and retracting the catheter from the urethra;
6) while using the same catheter, applying a stent of a selected length capable of local radial expansion in predetermined axial position onto said treatment balloon;
7) inserting the catheter thus prepared into said urethra;
8) expanding said positioning balloon and retracting the catheter to engagement of said balloon against the bladder neck to obtain correct position of the stent;
9) distending the treatment balloon by introducing a heated pressurized fluid therein to expand the stent at one or two ends of the stent so as to keep the stent in position in said urethra; and
10) removing fluid from the balloons and withdrawing the catheter from the urethra leaving the stent in position as implanted.

20. A method according to claim 19 wherein the length of the stent is adapted to the treatment length as measured in step 1.
